(19) United States
(12) Patent Application Publication
(10) Pub. No.: US 2021/0145497 A1
(43) Pub. Date: May 20, 2021

(54) MEDICAL TORQUE TIGHTENING DEVICE AND METHOD

(71) Applicant: HL TECHNOLOGY SA, La Chaux-De-Fonds (CH)

(72) Inventor: Mickael RISOLD, Cormondrèche (CH)

(21) Appl. No.: 16/627,833

(22) PCT Filed: Jul. 2, 2018
(86) PCT No.: PCT/CH2018/000028

§ 371 Date: Dec. 31, 2019

(30) Foreign Application Priority Data
Jul. 3, 2017 (CH) 864/17

(51) Int. Cl. A61B 17/88

(52) U.S. Cl. CPC A61B 17/8875; A61B 2090/031

(57) ABSTRACT

A torque tightening device having a gripping handle, an axial rod associated with the gripping handle and arranged to carry a tightening tool and containing a rigid body. A peripheral ring mounted on a fluted tube has a spiral thread, onto which is threaded an end tip of the body, in the form of a threaded bushing, the thread of which is complementary to the thread engaged with same. The peripheral ring is linked to the fluted tube and the end tip is linked to the body. The two threads link the fluted tube and the body. Rotation of the body causes the fluted tube to move in a linear manner. In practice, each “click” of the mechanism moves the fluted tube, for example, a fraction of a turn, and causes the two elements to move axially relative to each other, to disconnect the gripping handle and the axial rod.

This application is a National Stage completion of PCT/CH2018/000028 filed Jul. 2, 2018, which claims priority from Swiss patent application serial no. 864/17 filed Jul. 3, 2017.

### FIELD OF THE INVENTION

The present invention relates to a medical device for dynamometric tightening, of the screwdriver type, for the positioning and fixing of an object, in particular an orthopedic prosthesis, the tightening device comprising a gripping handle fitted coaxially at the periphery of a rear segment of a straight central rod, the straight central rod being able to rotate about its axis and having at its front end a tightening tool, the tightening tool being adapted to fit the object to be tightened, wherein the medical device for dynamometric tightening is provided with active mechanical components for transmitting a tightening torque to the gripping handle of the tightening tool.

The present invention also relates to a method for tightening or loosening a medical device for dynamometric tightening or loosening, of the screwdriver type, for the positioning and fixing of an object to be tightened, in particular an orthopedic prosthesis, the device comprising a tool for dynamometric tightening or loosening and the tool for dynamometric tightening or loosening being adapted to fit the object to be tightened.

### BACKGROUND OF THE INVENTION

Devices for dynamometric tightening are already known, notably instruments of the screwdriver type and torque wrenches, which are made in accordance with the concept of ratchets, comprising a rotating tightening tool with a faceted wall and comprising peripheral recesses that co-operate with spring-loaded balls or the like, whose function is to define a tightening torque.

The dynamometric tightening devices currently in use by practitioners have disadvantages, both structural and functional. Their design is complex and their assembly is a lengthy and delicate process, so the manufacturing costs are extremely high. For that reason they form part of a set of tools which a practitioner keeps carefully and re-uses for many applications, so that the practitioner has to sterilize the instrument, which must consequently be made of materials capable of resisting the high temperatures to which they are subjected during successive sterilizations.

Now, the new directives that have come into force in many countries impose upon manufacturers and users of medical instruments a “single use only” requirement in order to minimize the risks of contamination that may arise as a result of deficient sterilization. These rules are particularly restrictive, especially for manufacturers, in the sense that they imply that the manufacturer is responsible, even if the “single use” rule has not been complied with by the practitioner, if it has been possible to demonstrate that the device was not made in such manner that its construction precludes repeated use. That entails risks for the patient and implies responsibility for the entire chain of users, including the designer and manufacturer of the device.

The purpose of the present invention is to develop a device for dynamometric tightening, which complies strictly with the new norms in force and which introduces in the equipment concerned a type of progressive obsolescence, imposed in the context of respect for patient safety rules. Since re-use entails risks, the automatic suppression of the possibility of re-use eliminates those risks.

### SUMMARY OF THE INVENTION

The present invention aims to counter all the disadvantages mentioned above, by providing an instrument for dynamometric tightening or loosening, of the screwdriver type, in particular for the positioning of objects such as orthopedic prostheses, the instrument being of simple design, intended for single use, which allows the maker to avoid having to choose specific materials capable of resisting repeated sterilization operations, so that the cost of producing the instrument becomes particularly economical compared with the known instruments that have the same function.

This objective is achieved by the medical device for dynamometric tightening, which is moreover provided with integrated deactivation means for automatically deactivating the mechanical components designed to transmit the tightening torque after having been used a predetermined number of times, to couple mechanically the gripping handle with the axial rod bearing the dynamometric tightening tool in order to ensure the dynamometric tightening for positioning and fixing the object.

According to an advantageous embodiment, the predetermined number of times for mechanically coupling the gripping handle with the axial rod corresponds to a predefined number of active couplings of the gripping handle with the axial rod, carried out in order move the tightening tool in rotation.

Preferably, the integrated deactivation means for automatically deactivating the mechanical components that transmit the tightening torque comprise a first component operationally coupled to the gripping handle and a second component operationally coupled to the tightening tool by way of the axial rod, wherein the first component comprises a first axial displacement device, wherein the second component comprises a second axial displacement device, the first axial displacement device and the second axial displacement device being coupled together in such manner as to displace axially the second component each time the first component is moved in rotation, in order to decouple the gripping handle from the tightening tool in a predetermined way.

Advantageously, the first component is operationally coupled to the gripping handle, and consists of a tip with a narrowed section of a tubular portion fitted inside a rigid body attached to the gripping handle, and the second component is operationally coupled to the tightening or loosening tool and consists of a threaded annular ring attached to the axial rod carrying the tightening or loosening tool, the first axial displacement device consisting of a first peripheral thread formed on the tip, and the second axial displacement device consisting of a second, complementary peripheral thread formed on the threaded annular ring.

The gripping handle is advantageously made of a synthetic material, preferably overmolded with silicone over a rigid body.

The body is a rigid component with an anterior sector in the form of an open tube having a cylindrical cavity inside, arranged to receive components for transmitting a tightening torque of the gripping handle to the axial rod and to the tightening tool.

The components for transmitting a tightening torque advantageously comprise an at least partially cylindrical tube, fluted on the inside with longitudinal grooves all along its inner wall and located in the body, an interlocking mechanism for coupling the body with the longitudinal grooves, in order to drive the axial rod in rotation in the tightening direction, as long as the threshold torque applied to the gripping handle and to the dynamometric tightening tool does not exceed a predetermined value and to be detached relative to the longitudinal grooves when the belt of fins is turned in the tightening direction and the torque applied exceeds the predetermined value.

The rigid rod, which serves to carry the dynamometric tightening or loosening tool, is advantageously at least partially overmolded with a synthetic plastic material, for example a thermoplastic, to form a sleeve.

The belt of fins is preferably fitted on the sleeve, to which it is firmly attached.

According to the method for tightening a medical device for dynamometric tightening or loosening, the medical dynamometric tightening device is preferably provided with integral deactivation means for automatically deactivating mechanical components designed for the transmission of a tightening torque to the tool for dynamometric tightening or loosening, after having been used a predetermined number of times for the mechanical coupling of the gripping handle to an axial rod carrying the tool for dynamometric tightening or loosening.

The means for deactivating the gripping handle with the axial rod carrying the dynamometric tightening or loosening tool comprise a first screw-thread of a threaded tip that can move axially toward a second thread of a peripheral ring fitted onto a tubular section that carries the tightening and loosening tool, wherein the two threads are displaced and move apart linearly until they are disengaged, in a predetermined manner, during the use of the tightening and loosening mechanism of the medical device.

The displacement of the two threads takes place after a defined number of turns undergone by the medical tightening or loosening device by the gripping handle of the device.

The fins are preferably inclined relative to radial directions perpendicular to the axis of the axial rod, and have free ends in contact with the longitudinal grooves of the fluted tube on the inside, being configured to engage with the grooves when the fluted tube is turned in the direction that corresponds to tightening, and to disengage from the grooves when the tightening torque applied exceeds a predetermined value.

### BRIEF DESCRIPTION OF THE DRAWINGS

The present invention and its main advantages will become more evident from the description of various embodiments, with reference to the attached drawings, in which:

FIG. 1 represents an exterior perspective view of the medical dynamometric tightening device according to the invention,

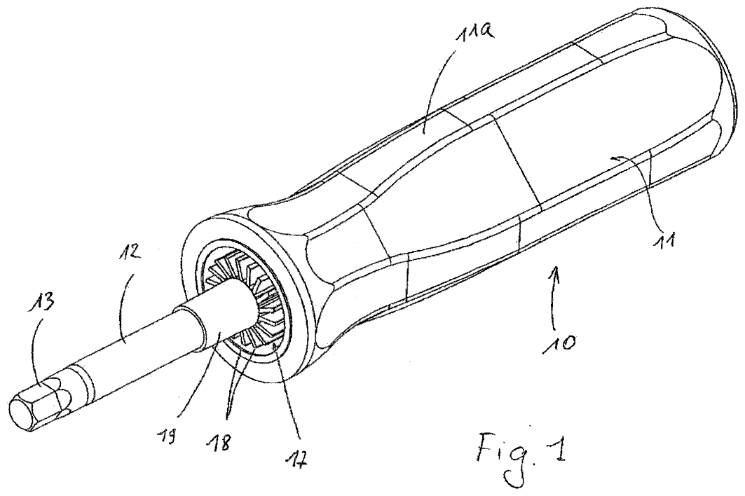

FIG. 2 is an exploded view of the medical dynamometric tightening device in FIG. 1, illustrating the main internal components,

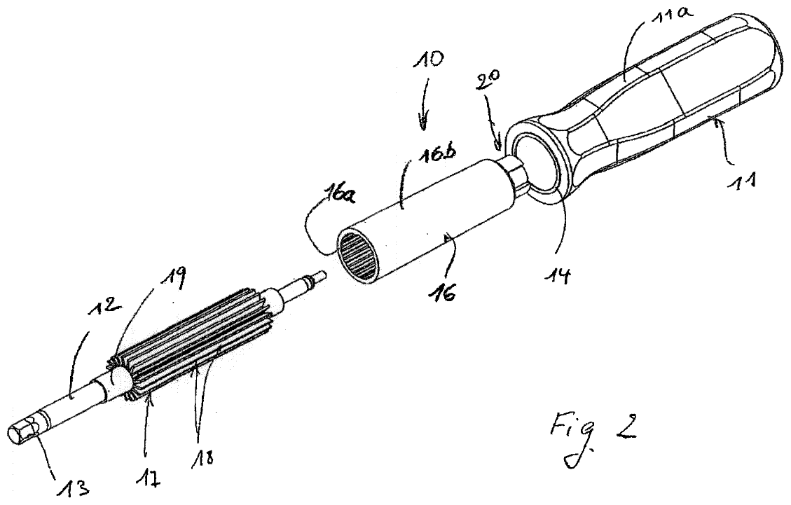

FIG. 3 shows a partially sectioned perspective view of the medical dynamometric tightening device of the preceding figures, illustrating some of the main internal components,

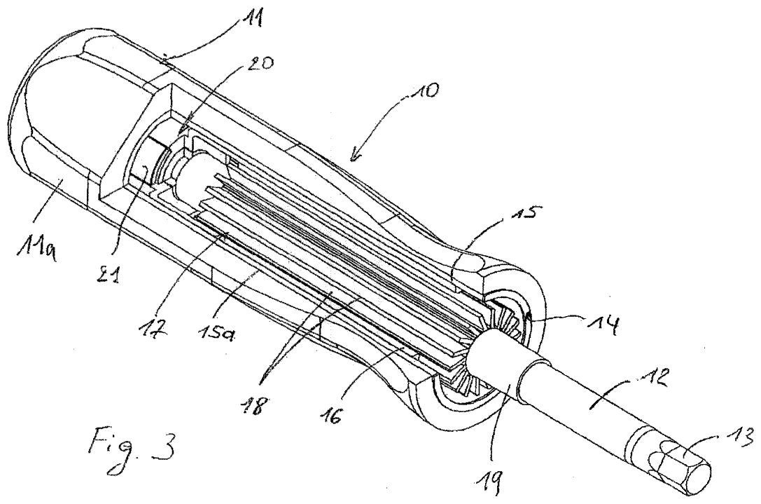

FIG. 4A is a view sectioned along an axial plane, showing the medical dynamometric tightening device of FIG. 1 to illustrate the main internal components,

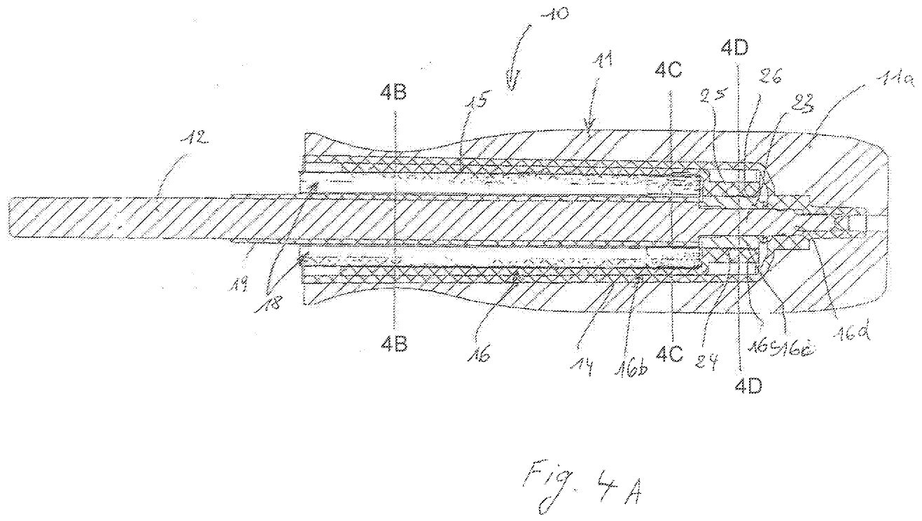

FIGS. 4B, 4C and 4D are cross-sectional views, respectively along the planes 4B-4B, 4C-4C and 4D-4D perpendicular to the longitudinal axis of the medical dynamometric tightening device according to the invention, and

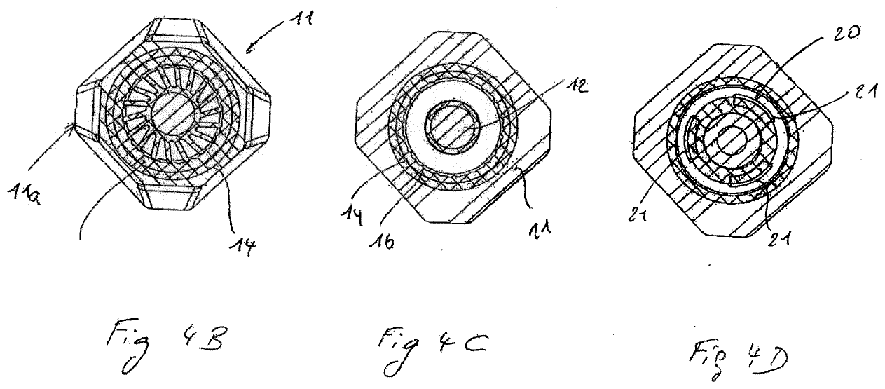

FIG. 5 shows a partially sectioned perspective view similar to that of FIG. 3, of the medical dynamometric tightening device in the preceding figures, which also illustrates certain internal components.

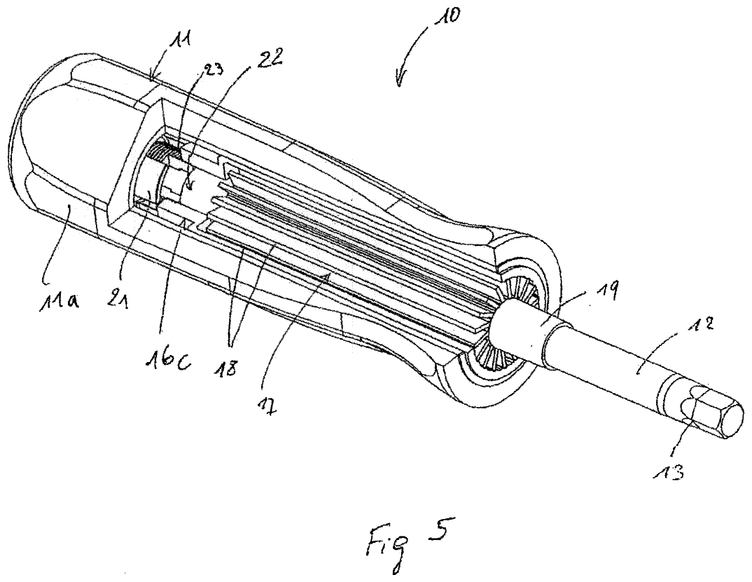

### DETAILED DESCRIPTION OF THE PREFERRED EMBODIMENTS

Referring to the figures, the dynamometric tightening device 10 illustrated resembles a screwdriver and comprises a gripping handle 11, an axial rod 12 associated with the gripping handle 11 and designed to carry at its free end a tightening tool 13, for example in the form of a tightening tip or socket. The gripping handle 11 serves to carry the tool and enables a user to apply a tightening torque to a tool for fixing an object. The tightening tool 13 is adapted to suit the application envisaged, so far as its profile and dimensions are concerned, in particular in order to ensure the positioning and tightening of the elements of orthopedic prostheses.

The gripping handle 11 consists of a handle 11a preferably made of a synthetic material, advantageously silicone, the handle being obtained by overmolding on top of a body 14, which is a rigid component that comprises an open tubular anterior section 15 and a cylindrical internal cavity 15a formed inside the anterior tubular section 15. Inside the body 14 and more precisely inside its internal cavity 15a is fitted a tube 16, fluted on the inside, which is configured to ensure the transmission of a tightening torque exerted by a user on the tightening handle 11a, and transmitted to the axial rod 12 and to the tightening tool 13. The fluted tube 16 is coupled to the body 14, which is itself firmly attached to the handle 11a of the gripping handle 11.

Inside the internal cavity 15a corresponding to the anterior tubular section 15 of the body 14, is arranged a belt 17 of longitudinal fins 18, the belt 17 being located coaxially relative to the axial rod 12, on a sleeve 19, preferably made of a synthetic plastic material, in particular thermoplastic, which is extruded directly onto a central portion of the axial rod 12. The longitudinal fins 18 are inclined relative to radial directions perpendicular to the axis of the axial rod 12. The free ends of the longitudinal fins 18 are in contact with the longitudinal fluting grooves 16a of the tubular anterior sector 16b of the internally fluted tube 16 and are configured so as to engage with the grooves 16a when the fluted tube 16 is turned, correspondingly to rotation in the same direction imposed by a user on the gripping handle 11. This engagement takes place when the first rotation direction is carried out toward the side toward which the longitudinal fins 18 are inclined. In that case the belt of fins 18 is entrained and carries with it the fluted tube 16 by engaging with the internal grooves 16a, and then communicates its rotational movement to the axial rod 12 and the tightening tool 13. When the fluted tube 16 turns in the opposite direction, namely in the direction opposite to the inclination of the longitudinal fins 18, the fins pass over the grooves 16a by virtue of their flexibility, without entraining the fluted tube 16.

The fluted tube 16 is connected rotationally fixed to the body 14 of the gripping handle 11 in order to transmit tightening torque from the handle 11a to the tightening tool, when the handle 11a is mechanically coupled to the axial rod 12. The design of the belt 17 of longitudinal fins 18 and the geometry of the grooves 16a determine a tightening torque threshold beyond which the fins can no longer engage with the grooves 16a and disengage when a tightening torque higher than the threshold is applied. It is this configuration which ensures the torque limiting function of the device 10. That function is known as such.

However. the coupling between the gripping handle 11 and the fluted tube 16 must be ensured in advance by way of the fluted tube 16, which is moved by virtue of the belt 17 of longitudinal fins 18. The transmission of the torque applied by a user to the gripping handle 11 takes place by way of an interlock mechanism 20 or the like, which as in the present case comprises, for example, three hooks 21 in the form of circular arc segments attached to the body 14. During operation the hooks 21, which effect the interlock, engage with the peripheral recesses 22 formed is a peripheral ring 23 mounted on a posterior sector 16c of the grooved tube 16. That posterior sector 16c has a diameter smaller than the diameter of the anterior tubular sector 16b and is extended by a thinner end sector 16d to ensure the centering and guiding of the fluted tube 16 relative to the body 14.

The peripheral ring 23 fitted on a posterior section of the fluted tube 16 has on its outer surface a screw thread 24, whose pitch is predetermined for reasons which will be explained below. Over the thread 24 is overmolded an end tip 25 of the body 14 in the form of a socket threaded on the outside, whose thread 26 is complementary relative to the thread 24 and is engaged therewith. The peripheral ring 23 is connected to the fluted tube 16 and the end tip 25 is connected to the body 14. The two threads 24 and 26 connect the two elements 23 and 25 and therefore the fluted tube 16 and the body 14. Rotation of the body 14 brings about a linear displacement of the fluted tube 16. In practice, each “click” of the mechanism moves the fluted tube, for example, by a fraction of a turn and causes a relative axial displacement of the two elements 23 and 25. The engagement of the hooks 21 decreases progressively until the moment when they move clear of the peripheral recesses 22, which results in decoupling the gripping handle 11 from the axial rod 12 and the tightening tool 13.

The special design feature of the instrument 10 is related to its mode of operation, which is characterized by what could be called a planned obsolescence, such that the instrument becomes inoperative after a predetermined number of rotations undergone by the axial rod 12, i.e. after a predetermined amount of utilization of the instrument.

The present invention is not limited to the embodiment described, but can undergo various modifications or variants. Designs of dynamometric tightening instruments that operate on the same principle but do not comprise integrated means for limiting their use in order to comply with safety criteria equivalent to those of the present instrument could be envisaged. Nevertheless, the present invention stands out from the main known instruments by virtue of this special design feature, so that it responds to the current requirements of the market.

1-13. (canceled)

14. A medical device for dynamometric tightening or loosening (10), of a screwdriver type, for positioning and fixing an object, the medical device for dynamometric tightening (10) comprising:
a gripping handle (11) mounted coaxially at a periphery of a posterior section of a central rectilinear axial rod (12),
the central rectilinear rod carrying a dynamometric tightening or loosening tool (13) at an anterior end thereof,
the dynamometric tightening or loosening tool (13) being adapted to fit the object to be tightened,
the medical device for dynamometric tightening (10) being provided with mechanical components (21, 22) designed to transmit a tightening torque applied to the gripping handle (11) of the tightening tool, and
the medical device for dynamometric tightening (10), in addition, being provided with integrated deactivation means (23, 25; 24, 26) for automatic deactivation of the mechanical components (21, 22) designed for the transmission of the tightening torque, after having been used a predetermined number of times, for mechanically coupling the gripping handle (11) to the axial rod (12) carrying the dynamometric tightening or loosening tool (13) to ensure the dynamometric tightening for positioning and fixing of the object.

15. The medical device for dynamometric tightening (10) according to claim 14, wherein the predetermined number of times for mechanically coupling the gripping handle (11) to the axial rod (12) corresponds to a predefined number of active couplings of the gripping handle (11) to the axial rod (12), carded out to rotate the dynamometric tightening or loosening tool (13).

16. The medical device for dynamometric tightening (10) according to claim 14, wherein the integrated deactivation means (23, 25; 24, 26) for the automatic deactivation of the mechanical components for transmitting the tightening torque comprises a first component (25) operationally coupled to the gripping handle (11) and a second component (23) operationally coupled to the dynamometric tightening or loosening tool (13), by way of the axial rod (12), and the first component (25) comprises a first axial displacement feature (24), the second component (23) comprises a second axial displacement feature (26), the first axial displacement feature (24) and the second axial displacement feature (26) being coupled with one another in such manner as to displace the second component (23) axially each time that the first component (25) is moved in rotation, with a view to decoupling the gripping handle (11) and the dynamometric tightening or loosening tool (13), in a predetermined manner.

17. The medical device for dynamometric tightening (10) according to claim 16, wherein the first component (25) operationally coupled to the gripping handle (11) comprises a narrower tip of a tubular component (16), fitted inside a rigid body (14) attached to the gripping handle (11), and the second component (23) operationally coupled to the dynamometric tightening or loosening tool (13) comprises a threaded annular ring (22) attached to the axial rod (12) carrying the dynamometric tightening or loosening tool (13), the first axial displacement feature (24) comprises a first peripheral thread formed on the tip, and the second axial displacement feature (25) comprises a complementary second peripheral thread formed on the threaded annular ring.

18. The medical device for dynamometric tightening (10) according to claim 17, wherein the gripping handle (11) is made of a synthetic material.

19. The medical device for dynamometric tightening (10) according to claim 17, wherein the body (14) is a rigid component with an anterior open tubular section (15), and has a cylindrical internal cavity (15a), designed to receive components for the transmission of a tightening torque from the gripping handle (11) to the axial rod (12) and to the dynamometric tightening tool (13).

20. The medical device for dynamometric tightening (10) according to claim 19, wherein the components for transmitting the tightening torque comprise an at least partially cylindrical tube (16), which is fluted on an inside with longitudinal grooves (16a) along an inside wall thereof and fitted in the body (14), an interlocking mechanism (20) for coupling the body (14) to the fluted tube (16), a belt (17) with fins (18) configured so as to co-operate with the longitudinal grooves (16a) with a view to causing the axial rod (12) to rotate in a direction of tightening, so long as a threshold of the torque applied to the gripping handle and to the dynamometric tightening tool does not exceed a predetermined value, and to disengage from the longitudinal grooves (16a) if the belt of fins (18) is turned in a tightening direction and the torque applied exceeds the predetermined value.

21. The medical device for dynamometric tightening (10) according to claim 14, wherein fins (18) are inclined relative to radial directions perpendicular to an axis of the axial rod (12) and have free ends in contact with longitudinal grooves (16a) of an internally fluted tube (16), and are configured to rest against the grooves (16a), when the fluted tube (16) is turned in a direction corresponding to tightening, up to a fixed torque limit.

22. The medical device for dynamometric tightening (10) according to claim 14, wherein the axial rod (12) that serves to carry the dynamometric tightening or loosening tool (13) is at least partially overmolded with a synthetic plastic material to form a sleeve (19).

23. The medical device for dynamometric tightening (10) according to claim 22, wherein a belt (17) of fins (18) is fitted on the sleeve (19), to which it is firmly attached.

24. The medical device for dynamometric tightening (10) according to claim 18, wherein the synthetic material is overmolded with silicone over a rigid body (14).

25. A method for tightening a medical device for dynamometric tightening or loosening (10), of a screwdriver type, for positioning and fixing an object, the device for dynamometric tightening (10) having a tool (13) for dynamometric tightening or loosening, and the dynamometric tightening or loosening tool (13) being adapted to fit the object to be tightened, the method comprising:
providing the medical device for dynamometric tightening (10) with integrated deactivation means for automatically deactivating mechanical components, designed to transmit a tightening torque to the dynamometric tightening or loosening tool (13), after having been used a predetermined number of times, to couple mechanically a gripping handle (11) to an axial rod (12) carrying the dynamometric tightening or loosening tool (13).

26. The method according to claim 25, wherein the deactivation means of the gripping handle (11), with the axial rod (12) carrying the dynamometric tightening or loosening tool (13), comprise a first thread on a threaded tip (25) that is axially movable on a second thread of a peripheral ring (23) fitted onto a tubular section (16) that carries the tightening and loosening tool (13), and the first and the second threads are displaced and move linearly apart until the first and the second threads disengage, in a predetermined manner, during use of the medical device for tightening and loosening.

27. The method according to claim 26, wherein the disengagement of the first and the second threads takes place after a defined number of turns undergone by the medical device for tightening or loosening, by the gripping handle of the device.
